(19) United States
(12) Patent Application Publication
(10) Pub. No.: US 2016/0361285 A1
(43) Pub. Date: Dec. 15, 2016

(54) METHOD FOR PURIFYING STEARIDONIC ACID

(71) Applicant: BIZEN CHEMICAL CO., LTD., Akaiwa-shi (JP)

(72) Inventors: Tadahiro Tsusima, Akaiwa-shi (JP); Yoshio Shimizu, Akaiwa-shi (JP)

(21) Appl. No.: 15/121,728

(22) PCT Filed: Feb. 13, 2015
(86) PCT No.: PCT/JP2015/000681

§ 371 Date: Aug. 25, 2016

(30) Foreign Application Priority Data
Feb. 28, 2014 (JP) 2014-039364

(51) Int. Cl. A61K 31/232; C07C 67/58; C12P 7/64; C07C 67/08

(52) U.S. Cl. CPC A61K 31/232; C07C 67/08; C07C 67/58; C12P 7/649

(57) ABSTRACT

The present invention relates to a method for producing and purifying SDA at high purity and/or high yield from a raw material such as anchovy oil. According to the present invention, when separating and purifying SDA from a raw material (e.g. anchovy oil) that includes SDA, high-quality, high-purity SDA is produced at a high recovery rate by using a combination of ethyl esterification and a silver nitrate treatment method. (1) A purification method selected from the group consisting of a thin-film vacuum superfractionation method, a simulated moving bed chromatography method, a urea addition method, and a molecular distillation method, and (2) an adsorbent treatment may be combined, as necessary.

### TECHNICAL FIELD

The present invention relates to the field of stearidonic acid purification.

### BACKGROUND ART

α-linolenic acid (ALA), stearidonic acid (SDA), eicosapentaenoic acid (EPA), and docosahexaenoic acid (DHA), which are ω3-based fatty acids contained in large quantity in fish oil, algae and the like, have the ability to improve hyperlipidemia and arteriosclerosis, and are also reported as having physiological activity such as immunity and inflammatory reaction. Thus, ω3 based fatty acids are materials that can be utilized in various applications (Non Patent Literature 1, Patent Literature 1).

However, low level of oxidation stability of EPA or DHA upon utilization thereof is problematic, such that application of EPA or DHA to food products is difficult in some cases. While ALA has a higher level of oxidation stability relative to EPA and DHA and ALA is a precursor of EPA in vivo, its level of physiological activity is lower than EPA or DHA and its lower efficiency of conversion to EPA is problematic (Non Patent Literature 2).

Meanwhile, SDA, which is an intermediate product generated when ALA is metabolized into EPA, has a high level of oxidation stability than EPA or DHA, and the efficiency of conversion into EPA is higher than ALA. Thus, it may be possible that SDA can be utilized as a precursor of EPA after intake into the body.

Natural sources of SDA include hemp, black currant, and echium seeds (Patent Literature 2). In recent years, SDA-containing soybeans have also been developed from genetic engineering (Patent Literature 3). Since anchovies are also a source of SDA, whose annual haul is about 1.0 million metric tons, they have been expected as a raw material for SDA. However, the SDA content is low, which is about 3 wt %, and purification thereof is difficult.

Known purification methods of fatty acids include molecular distillation, chromatography, silver nitrate treatment, and urea addition (Patent Literature 4, Patent Literature 5), which are available in industrial scale. However, there is no method that can efficiently purify SDA from raw materials such as anchovy oil.

### CITATION LIST

### Patent Literature

[PTL 1]: Japanese National Phase PCT Laid-open Publication No. 201.2-531911
[PTL 2]: Japanese National Phase PCT Laid-open Publication No. 2004-536801
[PTL 3]: Japanese National Phase PCT Laid-open Publication No. 2012-531911
[PTL 4]: Japanese Laid-Open Publication No. 11-209786
[PTL 5]: Japanese Laid-Open Publication No. 7-242895

### Non Patent Literature

[NPL 1] Mitsumasa MANKURA and Mitsu KAYAMA, “AA, EPA, DHA no Seirikino to Riyo” [Physiological function and utility of AA, EPA, DHA], AA, EPA, DHA−: highly unsaturated fatty acids, Ed. by Mitsu KAYAMA, KOUSEISHA KOUSEIKAKU Co., Ltd. (Tokyo), 1995, pp. 207-224
[NPL 2] Walker C G et al., “Stearidonic acid as a supplemental source of ω-3 polyunsaturated fatty acids to enhance status for improved human health.”, Nutrition, 2013, 29, pp. 363-369

### SUMMARY OF INVENTION

### Technical Problem

For this reason, there is a need for a method of producing/purifying SDA at a high level of purity and/or high yield from raw materials such as anchovy oil.

### Solution to Problem

It was discovered that high quality and high purity SDA can be produced at a high recovery rate by combining ethyl esterification (e.g., (a) ethyl esterification by lipolytic enzyme (e.g., lipase) treatment or (b) ethyl esterification by alkali treatment) with silver nitrate treatment for separating and purifying SDA from raw materials (e.g., anchovy oil) containing SDA, to complete the present invention. The present invention can obtain a purified product with SDA content of 80 wt % or greater by purifying SDA ethyl ester with (1) a purification method selected from the group consisting of thin film vacuum superfractionation, simulated moving bed chromatography, urea addition, and molecular distillation. The production method of the present invention may also use an adsorbent to remove impurities such as lipid peroxides, coloring components, or exogenous substances derived from the raw material.

Conventionally, a high level of purification of fatty acid ethyl ester such as SDA ethyl ester generally accompanies high temperature treatment such as distillation for a long period of time. Thus, there was a problem in terms of sensory evaluation such as order and color. Although the purification method of the present invention comprises a distillation step, an ethyl esterification reaction (e.g., enzymatic reaction or alkali treatment) and/or silver nitrate treatment is performed at a low temperature, such that high quality ethyl ester with SDA ethyl ester content of 80 wt % or greater can be obtained without any problems in terms of sensory evaluation such as order or color.

The present invention provides, for example, the following.

### (Item 1)

A method of producing stearidonic acid ethyl ester, comprising the steps of:

(a) treating a starting raw material comprising stearidonic acid with a lipolytic enzyme under an anhydrous condition to generate stearidonic acid ethyl ester and preparing a fraction comprising the stearidonic acid ethyl ester;
(b) mixing an aqueous medium solution comprising a silver salt with the fraction comprising the stearidonic acid ethyl ester to form a complex of the stearidonic acid ethyl ester and silver; and
(c) separating an aqueous medium phase comprising the complex of stearidonic acid ethyl ester and silver and adding a hydrophobic solvent to the separated aqueous medium phase to obtain a hydrophobic medium phase comprising stearidonic acid ethyl ester dissociated from the complex.

### (Item 2)

The method of item 1, further comprising the step of: (d) purifying the hydrophobic medium phase obtained in step (c) by a purification method selected from the group consisting of thin film vacuum superfractionation, simulated moving bed chromatography, urea addition, and molecular distillation.

### (Item 3)

The method of item 2, further comprising the step of: (e) treating a purified substance obtained by step (d) with an adsorbent to remove an impurity.

### (Item 4)

The method of item 1, wherein the lipolytic enzyme is a lipase derived from a microorganism of a genus selected form the group consisting of Alcaligenes, Candida, Pseudomonas, Penicillium, Aspergillus, Mucor, Rhizomucor, and Rhizopus.

### (Item 5)

The method of item 1, wherein a reaction condition of the lipolytic enzyme is the following:

a reaction temperature is 30 to 60° C.;

a reaction time is 6 to 72 hours; and

an amount of the lipolytic enzyme added is 50 to 1000 U per 1 g of the starting raw material.

### (Item 6)

The method of item 1, wherein a condition for forming the complex is the following:

a reaction temperature is 10 to 30° C.; and

a time period for having the complex form is 5 to 60 minutes; and

a condition for having stearidonic acid ethyl ester dissociate from the complex is the following:

a reaction temperature is 30 to 80° C.; and

a time period for having stearidonic acid ethyl ester dissociate from the complex is 5 to 90 minutes.

### (Item 7)

The method of item 2, wherein step (d) is thin film vacuum superfractionation, and an overhead degree of vacuum of a distillation apparatus is 0.2 mmHg or less and a distillation temperature is 150 to 200° C.

### (Item 8)

The method of item 3, wherein the adsorbent used in step (e) is selected from the group consisting of acid clay, activated clay, activated carbon, silicic acid, silica gel, zeolite, kaolin, perlite and alumina.

### (Item 9)

The method of item 8, wherein the adsorbent used in step (e) is degassed, nitrogen-replaced activated clay, an amount of the activated clay being 1 to 20 wt % of the weight of product obtained by step (d).

### (Item 10)

A composition comprising stearidonic acid ethyl ester obtained by the method of any one of items 3, 8, and 9, wherein

stearidonic acid ethyl ester content is 80 wt % or greater;

an acid value is 0.1 mg KOH/g or less;

a peroxide value is 2 meq/kg or less;

a color according to the Gardner scale is 4 or less; and

the composition is odorless to the sensory system;

### (Item 11)

A method of producing stearidonic acid ethyl ester comprising the steps of:

(a) ethyl esterifying a starting raw material comprising stearidonic acid to generate stearidonic acid ethyl ester and preparing a fraction comprising the stearidonic acid ethyl ester;
(b) mixing an aqueous medium solution comprising a silver salt with the fraction comprising the stearidonic acid ethyl ester to form a complex of the stearidonic acid ethyl ester and silver; and
(c) separating an aqueous medium phase comprising the complex of stearidonic acid ethyl ester and silver and adding a hydrophobic solvent to the separated aqueous medium phase to obtain a hydrophobic medium phase comprising stearidonic acid ethyl ester dissociated from the complex.

### (Item 12)

The method of item 11, wherein the ethyl, esterifying is performed by alkali treatment.

### (Item 13)

The method of item 11, further comprising the step of: (d) purifying the hydrophobic medium phase obtained in step (c) by a purification method selected from the group consisting of thin film vacuum superfractionation, simulated moving bed chromatography, urea addition, and molecular distillation.

### (Item 14)

The method of item 13, further comprising the step of:

(e) treating a purified substance obtained by step (d) with an adsorbent to remove an impurity.

### (Item 15)

The method of item 11, wherein a condition for forming the complex is the following:

a reaction temperature is 10 to 30° C.; and

a time period to have the complex form is 5 to 60 minutes; and

a condition for having stearidonic acid ethyl ester dissociate from the complex is the following:

a reaction temperature is 30 to 80° C.; and

a time to have stearidonic acid ethyl ester dissociate from the complex is 5 to 90 minutes.

### (Item 1.6)

The method of item 13, wherein step (d) is thin film vacuum superfractionation, and an overhead degree of vacuum of a distillation apparatus is 0.2 mmHg or less and a distillation temperature is 150 to 200° C.

### (Item 17)

The method of item 14, wherein the adsorbent used in step (e) is selected from the group consisting of acid clay, activated clay, activated carbon, silicic acid, silica gel, zeolite, kaolin, perlite and alumina.

### (Item 18)

The method of item 17, wherein the adsorbent used in step (e) is degassed, nitrogen-replaced activated clay, an amount of the activated clay being 1 to 20 wt % of a weight of the product obtained by step (d).

### (Item 19)

A composition comprising stearidonic acid ethyl ester obtained by the method of any one of items 14, 17, and 18, wherein

stearidonic acid ethyl ester content is 80 wt. % or greater;

an acid value is 0.1 mg KOH/g or less;

a peroxide value is 2 meq/kg or less;

a color according to the Gardner scale is 4 or less; and

the composition is odorless to the sensory system.

### Advantageous Effects of Invention

According to the present invention, high quality and high purity stearidonic acid (SDA) ethyl ester can be produced at a high recovery rate. The present invention provides a method of manufacturing SDA ethyl ester that is excellent in terms of sensory aspects such as color, order, and taste as well as safety aspects.

### DESCRIPTION OF EMBODIMENTS

The present invention is described hereinafter. Throughout the entire specification, a singular expression should be understood as encompassing the concept thereof in the plural form unless specifically noted otherwise. Further, the terms used herein should be understood to be used in the meaning that is commonly used in the art, unless specifically noted otherwise. Thus, unless defined otherwise, all terminologies and scientific technical terms that are used herein have the same meaning as the terms commonly understood by those skilled in the art pertaining to the present invention. In case of a contradiction, the present specification (including the definitions) takes precedence. As used herein, “wt %” is interchangeably used with “percent concentration by mass”

### DEFINITION OF TERMS

The definitions of the terms especially used herein are listed hereinafter.

As used herein, the term “starting raw material” refers to raw materials comprising stearidonic acid (SDA). Preferably, stearidonic acid (SDA) content of a starting raw material is 3 wt % or greater. A starting raw material may or may not be deacidified. A starting raw material may be a product of nature with or without crude purification. Preferably, the starting raw material of the present invention is oil and fat derived from fish, and more preferably anchovy oil.

As used herein, the term “anchovy” refers to fish of the Japanese anchovy family. Examples of anchovies of the present invention include, but are not limited to, anchoveta (Engraulis ringens), Japanese anchovy (Engraulis japonicus), European anchovy (Engraulis encrasicolus), and southern African anchovy (Engraulis capensis). Anchovies preferred in the present invention are anchovetas. The annual worldwide haul of anchovetas is about 10 million metric tons. The fat and oil thereof contain stearidonic acid.

As used herein, the term “glyceride” includes a component selected from the group consisting of triglyceride, diglyceride, and monoglyceride of fatty acids. In the present invention, “glyceride” does not include phospholipids or glycolipids unless specifically defined otherwise.

As used herein, the term “ethyl esterification” refers to a reaction for converting at least a part or the entire fatty acid (e.g., stearidonic acid (SDA)) contained in a starting raw material into stearidonic acid ethyl ester. Examples of ethyl esterification used in the present invention include, but are not limited to, lipolytic enzyme treatment and alkali treatment.

As used herein, the term “lipolytic enzyme” is an enzyme degrading lipids and is typically a lipase or cholesterol esterase. Preferably, the “lipolytic enzyme” of the present invention is a lipase. The “lipolytic enzyme” used in the present invention may be a natural enzyme or a recombinant enzyme. Further, the “lipolytic enzyme” used in the present invention may be in a solution form or an immobilized form. For example, a fatty acid (e.g., SDA) contained in a starting raw material is ethyl esterified in the presence of ethyl alcohol by a lipolytic enzyme.

The lipase used in the present invention is not particularly limited, but is typically a lipase derived from a microorganism of a genus selected form the group consisting of Alcaligenes, Candida, Pseudomonas, Penicillium, Aspergillus, Mucor, Rhizomucor, and Rhizopus. Alternatively, a lipase derived from the pancreas of a mammal such as a pig may be used. The commercially available lipases listed below can be used as the lipase of the present invention: (1) Meito Sangyo Co., Ltd.: lipase QLM (Alcaligenes), lipase PL (Alcaligenes), lipase PLC (Alcaligenes), lipase QLC (Alcaligenes), lipase OF (Candida); (2) Amano Enzyme Inc.: lipase AY (Candida), lipase G (Penicillium), lipase R (Penicillium). The preferred lipase is lipase QLM (Alcaligenes). The amount of enzyme used is 50 U to 1000 U, preferably 100 U to 900 U, more preferably 200 U to 800 U, and the most preferably 400 U to 600 U.

“Alkali treatment” of stearidonic acid as used herein refers to ethyl, esterification by alcoholysis, which reacts glyceride of a fatty acid or another ester in a starting raw material with ethyl alcohol in the presence of a basic catalyst. Examples of preferred basic catalyst include, but are not limited to, sodium methoxide, sodium ethoxide, sodium hydroxide, potassium hydroxide, lithium hydroxide, and magnesium hydroxide, and a hydroxide of an alkali metal or alkali earth metal therewith also corresponds thereto. The amount of catalyst used is 0.1% to 5% of the starting raw material and preferably 0.2% to 2%, but the amount is not limited thereto. A reaction temperature is preferably, but is not limited to, 20 to 90° C. and more preferably 40 to 70° C.

As used herein, the term “adsorbent” refers to a substance that; adsorbs impurities such as lipid peroxides, coloring components, or exogenous substances derived from the raw material generated during purification. Examples of adsorbent used in the present invention include, but are not limited to, acid clay, activated clay, activated carbon, silicic acid, silica gel, zeolite, kaolin, perlite and alumina.

As used herein, the term “purification” refers to any operation that increases the concentration of a substance subjected to purification.

As used herein, the term “SMB chromatography” refers to chromatography that is a separation method utilizing the principles of liquid chromatography in which multiple unit packing layers, filled with an adsorbent which has different selective adsorbing capability for a specific component in a raw material and for another specific component, are connected in series and the unit packing layer at the most downstream section and the unit packing layer of the most upstream section are connected to use a moving bed forming a circulating system with no ends. In the present specification, “SMB chromatography” and “simulated moving bed chromatography” are interchangeably used.

As used herein, the term “silver salt” refers to salts that are water-soluble silver compounds having the ability to form a complex with a π electron of a highly unsaturated fatty acid. Examples of silver salt used in the present invention include, but are not limited to, silver nitrate, silver perchlorate, silver tetrafluoroborate, and silver acetate. The silver salt used in the present invention is preferably silver nitrate.

A method of analyzing the composition of fatty acids and a method of measuring acid values, peroxide values (hereinafter, referred to as “POV”), and anisidine values used herein are well known and are as described, for example, in Standard Methods for the Analysis of Fats, Oils and Related Materials, 2003 (compiled by Japan Oil Chemist's Society).

### (Method of Production of the Present Invention)

The method of producing stearidonic acid ethyl ester of the present invention comprises:

(a) ethyl esterifying (e.g., by lipolytic enzyme treatment under an anhydrous condition or by alkali treatment) a starting raw material comprising stearidonic acid to generate stearidonic acid ethyl ester and preparing a fraction comprising the stearidonic acid ethyl ester;
(b) mixing an aqueous medium solution comprising a silver salt with the fraction comprising the stearidonic acid ethyl ester to form a complex of the stearidonic acid ethyl ester and silver; and
(c) separating an aqueous medium phase comprising the complex of stearidonic acid ethyl ester and silver and adding a hydrophobic solvent to the separated aqueous medium phase to obtain a hydrophobic medium phase comprising stearidonic acid ethyl ester dissociated from the complex.

### (Step a)

“Under an anhydrous condition” in lipolytic enzyme treatment is a condition under which moisture content is 50% or less, 40% or less, 30% or less, 20% or less, or 10% or less of a starting raw material. An anhydrous condition is typically a condition in the presence of an anhydrous ethanol solvent. The reaction temperature in an ethyl esterification reaction with a lipolytic enzyme (e.g., lipase) is 30 to 60° C. and preferably 40° C. The reaction time of an ethyl esterification reaction is 6 to 72 hours and preferably 24 hours.

A reaction condition for ethyl esterification by alkali treatment is, but not limited to, a condition under which a basic catalyst (e.g., sodium hydroxide) is added at 0.1 to 5%, preferably 0.5% to 3%, and more preferably 2% with respect to the starting raw material. The reaction temperature is preferably 20 to 90° C. and more preferably 40 to 70° C., but the reaction temperature is not limited thereto. While the stirring rate is preferably 50 to 500 rpm, the stirring rate is not limited thereto as long as the reactant is homogenous. The reaction time is preferably 15 to 120 minutes and more preferably 60 minutes, but the reaction time is not limited thereto.

If necessary, after an ethyl esterification reaction by lipolytic enzyme or alkali treatment, a hydrophobic solvent (e.g., n-hexane) is added and the mixture is left standing until two layers consisting of a hydrophobic solvent layer and an ethanol layer are formed, and the hydrophobic solvent layer comprising SDA ethyl ester is collected and washed with ethanol or water as needed. Furthermore, a glyceride fraction is removed by silica gel column chromatography if necessary.

### (Step b)

Examples of silver salt used in the present invention include, but are not limited to, silver nitrate, silver perchlorate, silver tetrafluoroborate, and silver acetate. The silver salt used in the present invention is preferably silver nitrate. The free fatty acids content in an aqueous medium solution comprising silver salt is not particularly limited, but is preferably 0.2 meq or less per 1 g of silver. The concentration of aqueous silver salt solution (e.g., aqueous silver nitrate solution) is preferably 20 to 70 wt % per reaction solution and most preferably 50 wt %. The temperature at which a complex of SDA ethyl ester and silver salt is formed is 10 to 30° C. and preferably 20° C. The time during which a complex is formed is 5 to 60 minutes and preferably 20 minutes.

### (Step c)

In the present invention, the temperature to have SDA ethyl ester dissociate from a complex is 30 to 80° C. and preferably 60° C. The time period for having ethyl ester dissociate from a complex is 5 to 90 minutes and preferably 30 minutes. Any hydrophobic solvent can be utilized as a solvent for having ethyl ester dissociate from a complex, but is preferably cyclohexane, n-hexane, chloroform, acetone, toluene or xylene. It is preferably n-hexane and/or cyclohexane.

### (Step d)

In the production method of the present invention, the hydrophobic medium phase obtained in the above-described step (c) may be purified by a purification method selected from thin film vacuum superfractionation, simulated moving bed chromatography, urea addition, and molecular distillation as needed.

### (d1: Thin Film Vacuum Superfractionation)

Thin film vacuum superfractionation is a method of separation utilizing the difference in boiling points of each component. For SDA ethyl ester, components with 18 carbon chains comprising SDA ethyl ester have a relatively low boiling point among fish oil fatty acids relative to components with 20 or more carbon chains. To purify SDA ethyl ester, the degree of vacuum in an distillation apparatus is typically set to 0.2 mmHg or less for multiple treatments at 150 to 200° C., but the condition is not limited thereto. The distillation temperature is preferably 174° C.

### (d2: Simulated Moving Bed Chromatography)

In a simulated moving bed chromatography (SMB chromatography), a raw material and eluent are supplied to a circulating system with no ends, and component X (i.e., component with weak affinity) moving at a high rate in a column (unit packing layer) and component Y (i.e., component with affinity) moving at a low rate in the column are each withdrawn from different positions. In addition, SMB chromatography sequentially moves the raw material supplying position, eluent supplying position, component X withdrawing position and component Y withdrawing position to the downstream side in the direction of fluid circulation while maintaining a certain positional relationship to materialize simulated operation to successively supply the raw material. As a result, SMB chromatography is an operational method that can move each component in a layer with maintaining the distribution thereof at approximately a certain width and maintaining the withdrawing position of each component as well as high purity/concentration.

### (d3: Urea Addition)

Urea addition is a method of purification utilizing the property of dissolved urea, which forms hexagonal adduct crystal while uptaking a straight chain molecule upon crystallization. For example, purification is performed by mixing and cooling a raw material and urea methanol solution to have a urea adduct taking in saturated fatty acid or monounsaturated fatty acid form, and filtering the resultant. Typically, n-hexane is extracted from a urea adduct and treated with silica gel, and then n-hexane is distilled to obtain an unsaturated fatty acid of interest.

### (d4: Molecular Distillation)

Molecular distillation can be performed, for example, by centrifugal molecular distillation or falling film molecular distillation. For example, falling film molecular distribution can perform treatment under a condition with 0.005 mmHg of degree of vacuum, 200° C. of evaporation surface temperature, and 30 g/L of flow rate to obtain a free fatty acid fraction as a fraction and glyceride fraction as a residue as described in Japanese Laid-Open Publication No. 2000-342291. For falling film molecular distillation, the degree of vacuum, evaporation surface temperature, and/or the amount of feed in molecular distillation operation can be appropriately changed by those skilled in the art according to the type of apparatus and raw material oil.

### (Step e)

The production method of the present invention may remove an impurity by treating a purified product obtained from the above-described step (d) with an adsorbent as needed. Examples of adsorbent used in the present invention include, but are not limited to, acid clay, activated clay, activated carbon, silicic acid, and alumina. The adsorbent is preferably activated clay or silica gel. Activated clay is preferably degassed, nitrogen-replaced activated clay, and the amount of activated clay added is 1 to 20 wt % of the product obtained by step (d).

A fraction (composition) comprising SDA ethyl ester obtained by the present invention typically has the following properties: stearidonic acid ethyl ester content is 80 wt % or greater; an acid value is 0.1 mg KOH/g or less; a peroxide value is 2 meq/kg or less; a color according to the Gardner scale is 4 or less; and the fraction has almost no odor to the sensory system.

### (Gardner Scale)

An example of evaluating the color of a purified specimen includes use of the Gardner scale. The Gardner scale is a method of placing a sample in a Gardner Holdt test tube and then comparing the color of the sample with standard colors of a standard color glass set to display a Gardner standard color number.

While the present invention is explained in detail in the following Examples and the like, the present invention is not limited thereto.

### EXAMPLES

### Example 1

After adding 500,000 U of lipase QLM (derived from Alcaligenes; Meito Sangyo Co., Ltd.) and 1000 mL of anhydrous ethanol with moisture content of 0.02 wt % or less to 1000 g of anchovy oil and stirring for 24 hours at 40° C., 2000 mL of n-hexane was added and left standing until two layers were formed with a separatory funnel. A hexane layer comprising an ethyl ester fraction was collected and then washed with ethanol and water. A glyceride fraction was removed by silica gel column chromatography using n-hexane:diethyl ether=95:5 (vol/vol) as an eluent to obtain 300.1 g ethyl ester. Purification by silica gel column chromatography was conducted in accordance with Inao ISHIZUKA, “Column chromatography”, Seikagaku [Biochemical] Data Book (I) Ed. by the Japanese Biochemical Society, Tokyo Kegaku Dojin, Tokyo, 1979, pp 869.

2500 g of aqueous 50 wt % silver nitrate solution (w/w) was added to the 300.1 g of anchovy oil ethyl ester and stirred at 20° C. to form a complex, which was left standing for 40 minutes with a separatory funnel. A complex fraction on the lower layer was then collected. 2500 g of hexane was added to the fraction and stirred at 60° C. to have the complex dissociate and dissolve ethyl ester to hexane. The hexane comprising ethyl ester was concentrated by a rotary evaporator to obtain 126.7 g of ethyl ester with SDA content of 21.3 wt %.

For subsequent purification by thin film vacuum superfractionation, 126.7 g of ethyl ester obtained by silver nitrate treatment was placed in a distillation apparatus and then distilled under the operating condition of 0.18 mmHg of overhead degree of vacuum and 173 to 175° C. of distillation temperature to obtain 21.3 g of main residual fraction of interest. The SDA content of the fraction was 83.2 wt %. The content of another main related substance, EPA, was 6.2 wt %, and DHA content was 4.0 wt %. After 5 wt % of activated clay was added to the ethyl ester obtained by thin film vacuum superfractionation and stirred for 30 minutes at 35° C., the resultant was filtered to obtain 20.6 g of ethyl ester with an acid value of 0.03 mg KOH/g, peroxide value of 0.9 meq/kg, anisidine value of 2.8, almost no odor in sensory evaluation, color of 3 (Gardner) and SDA content of 83.2 wt %. The recovery rate of SDA to the starting raw material was 55.3%.

The starting raw material, anchovy oil, and content of SDA, EPA and DHA after each treatment were as follows.

TABLE 1

| | SDA wt % | EPA wt % | DHA wt % |
|---|---|---|---|
| Anchovy oil | 3.1 | 18.3 | 9.0 |
| After lipase | 12.4 | 3.8 | 1.7 |
| treatment | | | |
| After silver | 21.3 | 7.7 | 4 .2 |
| nitrate | | | |
| treatment | | | |
| After vacuum | 83.2 | 6.2 | 4.0 |
| distillation | | | |

### Example 2

1000 g of anchovy oil was treated with lipase and silver nitrate as in Example 1 to obtain ethyl ester with stearidonic acid content of 21.3 wt %. The ethyl ester was then dissolved in methanol such that the ethyl ester would be 200 g/L. The sample was purified by setting a condition of 17.5 ml (17.5 mL/l-R/h) as the amount of raw material supply per 1 L of packing material per hour and 350 mL of flow per 1 L of packing material per hour with methanol as the eluent for purification by simulated moving bed chromatography for separation of two components using 6 ODS columns (φ10 mm×H 500 mm) to obtain ethyl ester with stearidonic acid content of 81.5 wt %. The content of another main related substance, eicosapentaenoic acid, was 8.4 wt % and the content of docosahexaenoic acid was 5.2 wt %. After 5 wt % of activated clay was added to the resulting ethyl ester and stirred for 30 minutes at 35° C., the resultant was filtered to obtain 19.6 g of ethyl ester with an acid value of 0.06 mg KOH/g, peroxide value of 1.8 meq/kg, anisidine value of 3.5, almost no odor in sensory evaluation, color of 3 (Gardner), and SDA content of 81.5 wt %. The recovery rate of SDA to the starting raw material was 51.5 wt %.

### Example 3

1000 g of anchovy oil was treated with lipase and silver nitrate as in Example 1 to obtain ethyl ester with stearidonic acid content of 21.3 wt %. The ethyl ester was then added and dissolved to a mixture of 400 g of urea and 1350 mL of ethanol by stirring for 60 minutes at 75° C. The mixture thereof was then cooled to room temperature and filtered to separate into the precipitate (urea adduct) and supernatant. The supernatant was collected and ethanol was removed. 200 mL, of water was added to dissolve the residue after removing ethanol. pH was adjusted to 3 to 4 with 6N hydrochloric acid to collect an oil component that separated and floated up to the top layer. The oil component was washed multiples times with water until the pH was neutral to remove urea, resulting in a urea-added purified product. The stearidonic acid content of the purified product was 80.4 wt %. The content of another main related substance, eicosapentaentaenoic acid, was 8.7 wt % and content of docosahexaenoic acid was 4.9 wt %. After 5 wt % of activated clay was added to the resulting ethyl ester and stirred for 30 minutes at 35° C., the resultant was filtered to obtain 17.3 g of ethyl ester with an acid value of 0.10 mg KOH/g, peroxide value of 1.9 meq/kg, anisidine value of 3.1, almost no odor in sensory evaluation, color of 3 (Gardner) and SDA content of 80.4 wt %. The recovery rate of SDA to the starting raw material was 44.9%.

### Example 4

1000 g of anchovy oil was added to 500 g of 2 wt % sodium hydroxide ethanol solution and stirred for 60 minutes at 200 rpm at 40° C. The reactant was then collected and washed multiple times with water until the pH was neutral to remove sodium hydroxide from the reactant, resulting in 807.3 g of anchovy oil ethyl ester. After the resulting anchovy oil ethyl ester was treated with silver nitrate as in Example 1 to obtain 337.5 g of ethyl ester with SDA content of 7.0 wt %, thin film vacuum superfractionation was performed to obtain 22.0 g of main residual fraction of interest. The fraction had SDA content of 80.8%. After activated clay was added to the resulting ethyl ester and stirred as in Example 1, the resultant was filtered to obtain 19.7 g of ethyl ester with an acid value of 0.02 mg KOH/g, peroxide value of 1.1 meq/kg, anisidine value of 3.6, almost no odor in sensory evaluation, color of 3 (Gardner) and SDA content of 80.2 wt %. The recovery rate of SDA to the starting raw material was 50.1%.

### Comparative Example 1

1000 g of anchovy oil was treated with lipase as in Example 1 to obtain 300.1 g of ethyl ester. The ethyl ester was added and dissolved into a mixture of 900 g of urea and 3000 ml, of ethanol by stirring for 60 minutes at 75° C. The mixture was then cooled to room temperature and filtered to separate into a precipitate (urea adduct) and supernatant. The supernatant was collected and ethanol was removed. 200 mL of water was added to dissolve the residual after removal of ethanol. pH was adjusted to 3 to 4 with 6N hydrochloric acid to collect an oil component that separated and floated up to the top layer. The oil component was washed multiple times with water until the pH was neutral to remove urea, resulting in a urea added purified product. The stearidonic acid content of this purified product was 24.7 wt %. The content of another main related substance, eicosapentaenoic acid, was 7.1 wt %, and content of docosahexaenoic acid was 3.6 wt %. After 5 wt % of activated clay was added to the resulting ethyl ester and stirred for 30 minutes at 35° C., the resultant was filtered to obtain ethyl ester with an acid value of 0.17 mg KOH/g, peroxide value of 2.8 meq/kg, anisidine value of 4.4, slight odor in sensory evaluation, and color of 5 (Gardner).

### Comparative Example 2

After 500 g of 1 wt % sodium hydroxide-ethanol solution was added to 1000 g of anchovy oil and stirred for 2 hours at 40′C, the oil layer in the top layer was collected. 250 g of water was added to the oil layer and stirred for 30 minutes at 70° C., and the water layer in the bottom layer was discarded. The same operation was repeated multiple times until the water layer was neutral to wash the oil layer. 50 g of anhydrous sodium sulfate was added to the oil layer and then filtered to obtain 947.2 g of anchovy oil ethyl ester. To subsequently purify by thin film vacuum superfractionation, the ethyl ester was placed in a distillation apparatus. Distillation was performed at an operation condition of 0.18 mmHg of overhead degree of vacuum and 173 to 175° C. of distillation temperature to obtain 85.7 g of main residual fraction of interest. The fraction had SDA content of 25.8 wt %. The content of another main related substance, EPA, was 41.2 wt %, and DHA content was 9.2 wt %. After 5 wt % of activated clay was added to the resulting ethyl ester and stirred for 30 minutes at 35° C., the resultant was filtered to obtain ethyl ester with an acid value of 0.06 mg KOH/g, peroxide value of 1.2 meq/kg, anisidine value of 3.9, almost no odor in sensory evaluation, and improved color. The result of analysis is shown in Table 2.

TABLE 2

| | | | | | | Comparative | Comparative |
|---|---|---|---|---|---|---|---|
| | | Example 1 | Example 2 | Example 3 | Example 4 | Example 1 | Example 2 |
| SDA content | wt % | 83.2 | 81.5 | 80.4 | 80.2 | 24.7 | 25.8 |
| Acid value | mg KOH/g | 0.03 | 0.06 | 0.10 | 0.02 | 0.31 | 0.06 |
| Peroxide | meq/kg | 0.9 | 1.8 | 1.9 | 1.1 | 3.9 | 1.2 |
| value | | | | | | | |
| Anisidine | — | 2.8 | 3.5 | 3.1 | 3.6 | 4.4 | 3.9 |
| value | | | | | | | |
| Odor | Sensory | Almost | Almost | Almost | Almost | Slight fish | Almost no |
| | evaluation | no odor | no odor | no odor | no odor | odor | odor |
| Color | Gardner | 3 | 3 | 3 | 3 | 5 | 5 |

### (Comparison of Examples and Comparative Examples)

Examples 1 to 3 are examples combining lipolytic enzyme and silver nitrate treatment. Example 4 is an example combining alkali, and silver salt treatment. Examples 1 and 4 further performed distillation purification and activated clay treatment. Example 2 further performed simulated moving bed chromatography purification and activated clay treatment. Example 3 further performed urea and activated clay treatment. The results of Examples 1 to 4 demonstrate that high quality and high purity stearidonic acid (SDA) ethyl ester can be produced at high recovery rate by combining ethyl esterification (e.g., (a) ethyl esterification by lipolytic enzyme treatment or (b) ethyl esterification by alkali treatment) with silver salt treatment.

The excellent effect of the present invention is further elucidated by comparing, for example, Example 3 with Comparative Example 1 (lipolytic enzyme treatment was performed, but not silver salt treatment) or Comparative Example 2 (neither lipolytic enzyme treatment nor silver salt treatment was performed) (by comparing especially SDA content, odor, and color).

As described above, the present invention is exemplified by the use of its preferred Embodiments. However, the present invention should not be limited to the Embodiments for interpretation. It is understood that the scope of the present invention should be interpreted solely based on the claims. It is understood that those skilled in the art can implement an equivalent scope from descriptions of the specific preferred embodiments of the present invention based on the description of the present invention and common general knowledge. Furthermore, it is understood that any patent, patent application, and references cited herein should be incorporated herein by reference in the same manner as the content are specifically described herein.

### INDUSTRIAL APPLICABILITY

High quality and high purity stearidonic acid (SDA) can be produced at a high recovery rate by the present invention. The present invention provides a method of manufacturing SDA ethyl ester that is excellent in terms of sensory aspects such as color, odor, and flavor as well as safety aspects. Highly pure SDA ethyl ester obtained by the present invention can be utilized as ω3 fatty acid ethyl ester in a broad range of industrial fields such as general food products, health-promoting food, cosmetic material, and is effective in maintaining or promoting human health.

1. A method of producing stearidonic acid ethyl ester, comprising the steps of:
(a) treating a starting raw material comprising stearidonic acid with a lipolytic enzyme under an anhydrous condition to generate stearidonic acid ethyl ester and preparing a fraction comprising the stearidonic acid ethyl ester;
(b) mixing an aqueous medium solution comprising a silver salt with the fraction comprising the stearidonic acid ethyl ester to form a complex of the stearidonic acid ethyl ester and silver; and
(c) separating an aqueous medium phase comprising the complex of stearidonic acid ethyl ester and silver and adding a hydrophobic solvent to the separated aqueous medium phase to obtain a hydrophobic medium phase comprising stearidonic acid ethyl ester dissociated from the complex.

2. The method of claim 1, further comprising the step of: (d) purifying the hydrophobic medium phase obtained in step (c) by a purification method selected from the group consisting of thin film vacuum superfractionation, simulated moving bed chromatography, urea addition, and molecular distillation.

3. The method of claim 2, further comprising the step of: (e) treating a purified substance obtained by step (d) with an adsorbent to remove an impurity.

4. The method of claim 1, wherein the lipolytic enzyme is a lipase derived from a microorganism of a genus selected form the group consisting of Alcaligenes, Candida, Pseudomonas, Penicillium, Aspergillus, Mucor, Rhizomucor, and Rhizopus.

5. The method of claim 1, wherein a reaction condition of the lipolytic enzyme is the following:
a reaction temperature is 30 to 60° C.;
a reaction time is 6 to 72 hours; and
an amount of the lipolytic enzyme added is 50 to 1000 U per 1 g of the starting raw material.

6. The method of claim 1, wherein a condition for forming the complex is the following:
a reaction temperature is 10 to 30° C.; and
a time period for having the complex form is 5 to 60 minutes; and
a condition for having stearidonic acid ethyl ester dissociate from the complex is the following:
a reaction temperature is 30 to 80° C.; and
a time period for having stearidonic acid ethyl ester dissociate from the complex is 5 to 90 minutes.

7. The method of claim 2, wherein step (d) is thin film vacuum superfractionation, and an overhead degree of vacuum of a distillation apparatus is 0.2 mmHg or less and a distillation temperature is 150 to 200° C.

8. The method of claim 3, wherein the adsorbent used in step (e) is selected from the group consisting of acid clay, activated clay, activated carbon, silicic acid, silica gel, zeolite, kaolin, perlite and alumina.

9. The method of claim 8, wherein the adsorbent used in step (e) is degassed, nitrogen-replaced activated clay, an amount of the activated clay being 1 to 20 wt % of the weight of product obtained by step (d).

10. A composition comprising stearidonic acid ethyl ester obtained by the method of any one of claims 3, 8, and 9, wherein
stearidonic acid ethyl ester content is 80 wt % or greater;
an acid value is 0.1 mg KOH/g or less;
a peroxide value is 2 meq/kg or less;
a color according to the Gardner scale is 4 or less; and
the composition is odorless to the sensory system;

11. A method of producing stearidonic acid ethyl ester comprising the steps of:
(a) ethyl esterifying a starting raw material comprising stearidonic acid to generate stearidonic acid ethyl ester and preparing a fraction comprising the stearidonic acid ethyl ester;
(b) mixing an aqueous medium solution comprising a silver salt with the fraction comprising the stearidonic acid ethyl ester to form a complex of the stearidonic acid ethyl ester and silver; and
(c) separating an aqueous medium phase comprising the complex of stearidonic acid ethyl ester and silver and adding a hydrophobic solvent to the separated aqueous medium phase to obtain a hydrophobic medium phase comprising stearidonic acid ethyl ester dissociated from the complex.

12. The method of claim 11, wherein the ethyl esterifying is performed by alkali treatment.

13. The method of claim 11, further comprising the step of: (d) purifying the hydrophobic medium phase obtained in step (c) by a purification method selected from the group consisting of thin film vacuum superfractionation, simulated moving bed chromatography, urea addition, and molecular distillation.

14. The method of claim 13, further comprising the step of: (e) treating a purified substance obtained by step (d) with an adsorbent to remove an impurity.

15. The method of claim 11, wherein a condition for forming the complex is the following:
a reaction temperature is 10 to 30° C.; and
a time period to have the complex form is 5 to 60 minutes; and
a condition for having stearidonic acid ethyl ester dissociate from the complex is the following:
a reaction temperature is 30 to 80° C.; and
a time to have stearidonic acid ethyl ester dissociate from the complex is 5 to 90 minutes.

16. The method of claim 13, wherein step (d) is thin film vacuum superfractionation, and an overhead degree of vacuum of a distillation apparatus is 0.2 mmHg or less and a distillation temperature is 150 to 200° C.

17. The method of claim 14, wherein the adsorbent used in step (e) is selected from the group consisting of acid clay, activated clay, activated carbon, silicic acid, silica gel, zeolite, kaolin, perlite and alumina.

18. The method of claim 17, wherein the adsorbent used in step (e) is degassed, nitrogen-replaced activated clay, an amount of the activated clay being 1 to 20 wt % of a weight of the product obtained by step (d).

19. A composition comprising stearidonic acid ethyl ester obtained by the method of any one of claims 14, 17, and 18, wherein
stearidonic acid ethyl ester content is 80 wt % or greater;
an acid value is 0.1 mg KOH/g or less;
a peroxide value is 2 meq/kg or less;
a color according to the Gardner scale is 4 or less; and
the composition is odorless to the sensory system.
